(19) United States
(12) Patent Application Publication
(10) Pub. No.: US 2012/0177700 A1
(43) Pub. Date: Jul. 12, 2012

(54) Nanonized Iron Compositions and Methods of Use Thereof

(76) Inventors: Mir A. Imran, Los Altos Hills, CA (US); Mir Hashim, Fremont, CA (US); Lu Wang, San Jose, CA (US); Sanjay Patel, Palo Alto, CA (US); Joel M. Harris, Mountain View, CA (US); Phil Morgan, San Antonio, TX (US)

(21) Appl. No.: 13/338,210

(22) Filed: Dec. 27, 2011

Related U.S. Application Data
(60) Provisional application No. 61/460,205, filed on Dec. 27, 2010.

(51) Int. Cl. A61K 9/00; A61K 33/42; B82Y 5/00

(52) U.S. Cl. 424/400; 424/604; 977/773; 977/915

(57) ABSTRACT

Embodiments of the invention provide nanonized iron compositions for treatment of iron deficiency such as iron deficiency anemia. Many embodiments provide nanonized iron compositions which are sized to minimize adverse reaction such as immune response, adverse GI reaction and allergic reaction to iron compound included in the composition. The nanonized iron compositions can be used in a variety of drug delivery forms, including an oral dosage form, a transdermal patch, in an intravenous solution or in a dialysate for treatment of a patient with chronic kidney disease (CKD). Embodiments of the invention also provide methods of using the nanonized iron compositions for the treatment of iron deficiency in a patient in need thereof including patients with iron deficiency anemia and CKD.

### RELATED APPLICATIONS

This application claims the benefit of priority of Provisional U.S. Patent Application Ser. No. 61/460,205, entitled “Nanonized Iron Compositions and Methods of Use Thereof”, filed Dec. 27, 2010; which is fully incorporated by reference herein for all purposes.

### FIELD OF THE INVENTION

Embodiments described herein relate to nanonized iron compositions. Additional embodiments relate to the use of the nanonized iron compositions for the treatment of iron deficiency, such as in iron deficiency anemia.

### BACKGROUND

Nutrient deficiency is a world wide heath problem. Iron deficiency in particular is the most common form of nutritional deficiency in the world, affecting over 3 billion people in the Third World alone. It is associated with a number of diseases and conditions and is blamed for 100,000 maternal deaths during childbirth each year as well as 134,000 deaths among children. It is also co-morbid with a number of parasitic infections and is a key underlying factor in impaired mental development in children.

Iron deficiency anemia is one of the more serious conditions caused by iron deficiency. It is an advanced stage of iron deficiency and occurs when the dietary intake or absorption of iron is insufficient, and hemoglobin, which contains iron, cannot be formed.

Children and pre-menopausal women are the two groups most prone to developing this disease. The principal cause of iron deficiency anemia in premenopausal women is blood lost during menses. Causes in children include malnutrition during pregnancy, malnutrition, premature birth, GI bleeding, and parasitic infection.

The typical form of treatment for iron deficient anemia includes oral or intravenous delivery with various ferrous compounds. However, both of these treatments have a number of limitations. Oral iron preparations in particular have many disadvantages. First and foremost, they cause gastrointestinal side effects including nausea, bloating, constipation, and diarrhea. This leads to discontinuation of iron supplementation in approximately 40-66% of the patients taking such supplements. Furthermore, the absorption of iron is variable and affected by the oral ingestion of other compounds. For example, oral ingestion of food products reduces iron absorption by approximately 50%, which is problematic since many patients take iron with food in order to reduce the gastrointestinal side effects.

Second, many drugs are known to reduce iron absorption. For example, oral ingestion of antacids and other drugs that reduce stomach pl-I is known to decrease iron absorption. In turn, oral ingestion of iron also reduces the absorption of many drugs, including antibiotics. Additionally, many conditions associated with iron deficiency anemia respond poorly to oral iron supplementation because iron cannot be properly absorbed through the cells of the gastrointestinal system. This is especially true of certain inflammatory conditions of the bowel, such as Crohn's disease. Additionally, diseases associated with functional iron deficiency, such as the anemia of renal failure, are also associated with limited absorption of orally administered iron. This is also true of many other so-called “inflammatory conditions” associated with functional iron deficiency, such as those associated with rheumatoid arthritis and other autoimmune diseases, as well as anemia secondary to cancer or cancer chemotherapy treatment. This is especially true in patients with these conditions who are treated with erythropoietin, who have considerably increased demands for iron.

Intravenous (IV) administration also has a number of limitations. These include pain and infection at the injection site, the requirement to be connected to an IV drip for a prolonged period of administration (to reduce the risk of anaphylaxis), and the requirement to mix, store and administer the medication in liquid form using sterile technique. The latter can be particularly problematic in third world countries where adequate refrigeration and sterile needles are not readily available, limiting shelf life and exposing the patient to infection. Also, IV administration can include several risk factors including anaphylaxis, and cardiovascular complications.

Thus, there is a need for improved methods of drug delivery for the treatment of iron deficiency including anemia and other related medical conditions which can extend shelf life and are more easily used in settings lacking refrigeration or sterile medical supplies.

### BRIEF SUMMARY

Embodiments described herein provide nanonized iron compositions for treatment of iron deficiency, including the treatment of iron deficiency anemia (sometimes referred to herein as “anemia”) and related conditions. Any appropriate method can be used to make the nanonized iron composition, including for example, the Rapid Expansion of Supercritical Solutions (RESS) process, the Supercritical Anti-Solvent (SAS) method, and the Particles from Gas Saturated Solutions (PGSS) method. Embodiments of the nanonized iron compositions can be in any appropriate drug delivery form, including for example, an oral dosage form, a transdermal patch, or in a dialysate for treatment of a patient with chronic kidney disease. Still other forms known in the pharmaceutical and drug delivery arts are also contemplated. In particular embodiments, the nanonized iron compositions can be configured to dissolve in an aqueous solution disposed in a transdermal patch used for passive or active transport (e.g., by iontophoresis) of the iron composition across the skin of a patient being treated for iron deficiency. Further, the nanonized iron compositions can be configured to dissolve in situ in the patch when the aqueous solution is added to or released within the patch to achieve a selected iron concentration and to do so with minimal or no mixing of the solution.

Various embodiments of the invention provide nanonized iron compositions wherein the iron-containing nanoparticles have a diameter of 1000 nm or less, or 500 nm or less, or 250 nm or less, or 100 nm or less, or 50 nm or less. Additional embodiments provide nanonized compositions in which 50% or more of the particles have a diameter within 1 to 1000 nm, or within 1 to 500 nm, or within 1 to 250 nm, or within 1 to 100 nm, or within 1 to 50 nm, or within 10 to 1000 nm, or within 10 to 500 nm, or within 10 to 250 nm, or within 10 to 100 nm, or within 10 to 50 nm, or within 50 to 1000 nm, or within 50 to 500 nm, or within 50 to 250 nm, or within 100 to 1000 nm, or within 100 to 500 nm, or within 100 to 250 nm. One or more these particle sizes (or others described herein) can be selected such that when the particles are added to a parenteral or other solution for administration to the patient the nanonized compositions dissolve sufficiently in the solution so that no filtering of the solution is needed to prevent an immune response such as a pyrogenic reaction due to any un-dissolved particles.

Still additional embodiments provide nanonized iron compositions wherein the iron-containing nanoparticles have a diameter of 50,000 nm or less, or 25,000 nm or less, or 10,000 nm or less, or 5000 nm or less, or 2500 nm or less. Further embodiments provide nanonized compositions in which 50% or more of the particles have a diameter within 100 to 50,000 nm, or within 100 to 25,000 nm, or within 100 to 10,000 nm, or within 100 to 5000 nm, or within 1000 to 50,000 nm, or within 1000 to 25,000 nm, or within 1000 to 10,000 nm, or within 1000 to 5000 nm, or within 5000 to 50,000 nm, or within 5000 to 25,000 nm, or within 5000 to 10,000 nm, or within 10,000 to 50,000 nm, or within 10,000 to 25,000 nm.

Yet additional embodiments provide nanonized iron compositions wherein the iron-containing nanoparticles have a diameter of 500,000 nm or less, or 250,000 nm or less, or 100,000 nm or less, or 50,000 nm or less, or 25,000 nm or less. Still further embodiments provide nanonized compositions in which 50% or more of the particles have a diameter within 1000 to 500,000 nm, or within 1000 to 250,000 nm, or within 1000 to 100,000 nm, or within 1000 to 50,000 nm, or within 10,000 to 500,000 nm, or within 10,000 to 250,000 nm, or within 10,000 to 100,000 nm, or within 10,000 to 50,000 nm, or within 50,000 to 500,000 nm, or within 50,000 to 250,000 nm, or within 50,000 to 100,000 nm, or within 100,000 to 500,000 nm, or within 100,000 to 250,000 nm.

Other embodiments of the invention provide for nanonized iron compositions having a variety of particles shapes including, for example, spherical, aspherical and tubular. Related embodiments provide for methods for fabrication of such shapes. One or more of these shapes can include a porous surface to increase surface area for contact with liquid for faster rates of dissolution in a parenteral or other solution and/or in bodily fluids for faster bioavailability. Also, one or more these shapes can comprise an outer shell (which may be porous surrounding a hollow inner cavity so as to provide for additional surface area for contact with liquid so as to enhance rates of dissolution and bioavailability. Specific preferred embodiments provide nanonized iron composition comprising hollow nanotubes. Related embodiments provide for methods of fabrication of such nanotubes. The length and other dimensions of the nanotubes can be configured to have various properties and/or perform one or more functions. For example, the iron-containing nanotubes can be configured to be spun or otherwise propelled by the magnetic force from an external magnet that is spinning or moving in another manner. This allows the nanotubes to act as magnetic spin bars when they are added to a solution containing nanonized iron particles to facilitate aseptic mixing of the solution so as to increase rates and completeness of dissolution of the nanonized iron including that of iron-containing nanotubes. Other embodiments of the nanonized iron compositions can also be sized or other configured to spin or otherwise be propelled by an external magnetic force so as to facilitate mixing of a solution containing the nanonized iron compositions.

Other functions of the iron-containing nanotube are also contemplated such as the ability to attach a functional group to an end of the nanotube (e.g., a polypeptide) so as to be attach to a particular cell type and thus deliver iron directly to that cell. For example, in one or more embodiments, functional groups can be attached to the iron-containing nanotubes so that they attach to or are otherwise taken up monocytes, macrophages or other RE cells in the reticuloendothelial (RE) system so as to directly deliver iron to those cells which recycle iron in the body. In related embodiments, the nanotubes can have a length or other dimensional feature which predisposes them to be phagocytized or otherwise absorbed by RE cells.

Still other embodiments provide a method for the treatment of iron deficiency in a patient in need thereof. The method comprises administering to the patient a therapeutically effective amount of the nanonized iron composition, for example, in an oral dosage form, a transdermal patch, in an intravenous solution or in a dialysate for treatment of a patient with chronic kidney disease. Many embodiments a provide such a method whereby the nanoparticles of the nanonized iron composition are sized or otherwise configured to minimize an adverse reaction of the patient to administration of an iron compound included in the nanonized iron composition. This includes minimizing the adverse reaction when the composition is administered for treatment of the patient's iron deficiency over a prolonged period including periods of weeks or even months. Such treatment can include that for iron deficiency anemia to increase the number of the patient's red blood cells or other store of iron in the patient's body such as ferritin iron complexes. The adverse reactions minimized can include one or more of an immune response, pyrogenic reaction, adverse GI (gastro intestinal) reaction (e.g., diarrhea, cramping or constipation), or an allergic reaction. Further as described herein, particles sizes can be selected for the nanonized iron-containing compositions such that when particles of the nanonized iron-containing compositions are added to a parenteral or other solution to be administered to the patient, the particles dissolve sufficiently in the solution such that no filtering of the solution is needed to remove any undissolved particles which may cause adverse reaction such as pyrogenic reaction or other immune response.

Still other embodiments of the invention provide a kit for the administration of nanonized iron compositions to a patient in need thereof comprising one or more embodiments of the nanonized iron compositions described and instructions for administering those compositions to a patient in need thereof. The instructions can include directions for the dosage and other details for administering the nanonized iron compositions so as to increase and/or replenish an iron store of the patient such as that found in the patient's red blood cells and that complexed to ferritin in the patient's cells throughout their body including for example, cells found in bone, liver and spleen tissue. Related embodiments provide a method for using the instructions to administer nanonized iron compositions to increase and/or replenish an iron store of a patient (e.g., red blood cells, etc.). The kit may also include instructions for screening patients who may have an immune response to such nanonized iron compositions and then selecting nanonized iron compositions having a particle size or range of particle sizes which minimize such a response. Related embodiments provide a method for using the instructions for screening a patient who may have an immune response to such nanonized iron compositions and then selecting compositions which minimize such a response.

In some embodiments, the kit can comprise a plurality of groups of nanonized iron compositions, with each group including nanonized iron compositions having a different particle size or particle size range from that in other groups. Typically, the groups of nanoparticles will be disposed in separate containers such as vials, ampules etc., but not necessarily. The instructions in such embodiments can include directions for selecting and/or selecting and mixing nanonized iron compositions from one or more of the containers so as to yield a nanonized iron composition(s) having a particle size or range of particle size or range of particle sizes which minimizes the immune response of a patient to such compositions. Related embodiments provide a method for using the instructions to select, prepare and/or administer nanonized iron compositions to a patient in need thereof which minimizes the immune response of the patient to such compositions.

Further details of these and other embodiments and aspects of the invention are described more fully below.

### DETAILED DESCRIPTION OF THE INVENTION

Many embodiments described herein provide nanonized iron compositions (also referred to herein as nanonized iron-containing compositions) which comprise one or more nanonized iron compounds (also referred to herein as nanonized iron-containing compounds.). As used herein, a nanonized composition is that containing particles having at least one dimension (e.g., diameter) in the range of about 1 to about 1000 nm. Such particles are referred to herein as nanoparticles and/or nano-sized particles. The nanoparticles comprising the nanonized iron-containing compositions may have a variety of shapes including for example, spherical, asphereical, tubular etc. Additionally, in various embodiments, the nanoparticles may be porous and/or hollow so as to increase surface area for contact with fluids for dissolution including bodily fluids (e.g., blood, interstitial fluid, intestinal fluids, etc.) as well as fluids used to make iron-containing parenteral and other solutions (e.g., solutions in a transdermal patch) administered to a patient.

Embodiments of the nanonized compositions may be produced by any appropriate method. Exemplary methods include the Rapid Expansion of Supercritical Solutions process (RESS process), the Supercritical Anti-Solvent method (SAS method), and the Particles from Gas Saturated Solutions method (PGSS method) and combinations thereof. Other methods for producing nanonized compositions known in the art are also contemplated.

In one or more embodiments, a nanonized iron composition can be produced by the RESS process. In the case of RESS, a supercritical fluid is used to dissolve a solid material (e.g., an iron composition) under high pressure and temperature, thus forming a homogeneous supercritical phase. Thereafter, the solution is expanded through a nozzle and small particles are formed. At the rapid expansion point which is at the opening of the nozzle, there is a sudden pressure drop that forces the dissolved material (the solid) to precipitate out of the solution. The crystals that are instantly formed enclose a small amount of the solvent that, due to the expansion, changes from supercritical fluid to its normal state (usually gas), thus breaking the crystal from inside-out. At the same time, further reduction of size is achieved while the forming and breaking crystals collide with each other at the vicinity of the nozzle. The particles that are formed this way have a diameter of a few hundreds of nanometers. To aid in the formation of nanonized iron compositions by the RESS process, one or more stabilizing agents or surface agents may be included to aid in the production of nanoparticles. See, for example, U.S. Pat. Nos. 6,682,761, 6,974,593 and 7,754,243, (the contents of which are hereby incorporated by reference herein for all purposes) for further description of such stabilizing and surface agents and their uses. The particle size can be controlled by one or more of the nozzle size, nozzle pressure and stabilizing and/or surface agents.

In various embodiments, particles of an iron composition treated by the RESS process will typically have a diameter of about 1000 nm or less, or 500 nm or less, or 250 nm or less, or 100 nm or less, or 50 nm or less. Exemplary nanonized compositions include those in which 50% or more of the particles have a diameter within 1 to 1000 nm, or within 1 to 500 nm, or within 1 to 250 nm, or within 1 to 100 nm, or within 1 to 50 nm, or within 10 to 1000 nm, or within 10 to 500 nm, or within 10 to 250 nm, or within 10 to 100 nm, or within 10 to 50 nm, or within 50 to 1000 nm, or within 50 to 500 nm, or within 50 to 250 nm, or within 100 to 1000 nm, or within 100 to 500 nm, or within 100 to 250 nm.

In other embodiments, nanonized iron compositions can be produced by the SAS method. In the SAS method, a solid material (e.g., an iron composition) is dissolved in an organic solvent and a supercritical fluid is then also forced by means of pressure to dissolve in the system. In this way, the volume of the system is expanded, thus lowering the density, and therefore also the solubility of the material of interest is decreased. As a result, the material precipitates out of the solution as a solid with a very small particle diameter.

In various embodiments, particles of an iron composition treated by the SAS process will typically have a diameter of about 50,000 nm or less, or 25,000 nm or less, or 10,000 nm or less, or 5000 nm or less, or 2500 nm or less. Exemplary nanonized compositions include those in which 50% or more of the particles have a diameter within 100 to 50,000 nm, or within 100 to 25,000 nm, or within 100 to 10,000 nm, or within 100 to 5000 nm, or within 1000 to 50,000 nm, or within 1000 to 25,000 nm, or within 1000 to 10,000 nm, or within 1000 to 5000 nm, or within 5000 to 50,000 nm, or within 5000 to 25,000 nm, or within 5000 to 10,000 nm, or within 10,000 to 50,000 nm, or within 10,000 to 25,000 nm.

In still other embodiments, nanonized iron compositions can be produced by the PGSS method. In the PGSS method, a solid material (e.g., an iron composition) is melted and the supercritical fluid is dissolved in it, like in the case of the SAS method. However, in this case, the solution is forced to expand through a nozzle, and in this way, nanoparticles are formed. The particle size can be controlled by one or more of the nozzle size, nozzle pressure and selected supercritical fluid.

In various embodiments, particles of an iron composition treated by the PGSS process will typically have a diameter of about 500,000 nm or less, or 250,000 nm or less, or 100,000 nm or less, or 50,000 nm or less, or 25,000 nm or less, 10,000 nm o, 1000 nm or less, or 500 nm or less, or 100 nm or less. Exemplary nanonized compositions include those in which 50% or more of the particles have a diameter within 10 to 100 nm, or within 100 to 250 nm, or within 100 to 500nm, or within 250nm to 500nm, or within 500 to 1000, nm or within, 1000 to 500,000 nm, or within 1000 to 250,000 nm, or within 1000 to 100,000 nm, or within 1000 to 50,000 nm, or within 1000 to 10,000 nm, or within 10,000 to 500,000 nm, or within 10,000 to 250,000 nm, or within 10,000 to 100,000 nm, or within 10,000 to 50,000 nm, or within 50,000 to 500,000 nm, or within 50,000 to 250,000 nm, or within 50,000 to 100,000 nm, or within 100,000 to 500,000 nm, or within 100,000 to 250,000 nm.

Various embodiments of invention also contemplate compositions which comprise combinations of one or more size distributions of nanonized iron particles. For example, iron-containing particles having 50% or more of the particles with a diameter within 50 to 500 nm could be combined with iron-containing particles having 50% or more of the particles with a diameter within 5000 to 50,000 nm. In general, the smaller the particle size, the more quickly the iron becomes bioavailable (e.g., due to faster rates of dissolution in body tissue fluids, such as in the GI track, saliva, fluid coating mucous membranes interstitial fluid or blood). Thus, by combining nanonized iron-containing particles having one or more size distributions one can tailor the pharmacokinetic properties of the compositions such that some of the iron is bioavailable quickly and some of the iron becomes bioavailable on a delayed or more prolonged basis. In particular embodiments, the size distributions of nanonized iron compositions can have a biomodal size distribution configured to have some of the iron become bioavailable after a first time period and the remainder after a second longer time period. In this way, embodiments of nanonized iron compositions can be used to achieve a more controlled delivery of bioavailable iron to the patient over an extended period of time and in turn, improved treatment for iron deficiency anemia and other forms of iron deficiency. In various embodiments, this tailored bioavailability can be adapted for use in one or more forms of administration of the iron-containing nanoparticles including for example, oral, sublingual inhaled/nasal, dermal/sub-dermal (in solid or liquid form), parenteral (including intravenous, dialysate, intramuscular, etc.) and other forms described herein. In particular embodiments the nanonized iron compositions can have a biomodal or other size distribution configured for oral and/or sublingual administration so that a portion of the iron becomes available after the first period and the remainder after a second longer period. The first period may correspond to a period as soon as ten minutes, or as soon as five minutes or even as soon as one minute. The second period may correspond to a period as soon as 60 minutes, or as soon as 30 minutes, or as soon as 20 minutes or even as soon as ten minutes, with longer and shorter periods contemplated. In these and related embodiments, the nanoparticles which become bioavailable during the first time period can be sized and other otherwise configured to dissolve in the saliva so as to be absorbed sublingually and those which become available during the second period can be sized and otherwise configured to not substantially be dissolved before being swallowed such that they dissolve in the GI fluids in the stomach and small intestine and are predominantly absorbed into the small intestine. In other embodiments, the nanoparticles can have a biomodal or other distribution configured for parenteral administration (either in solid or liquid form, the later including intravenous solutions and dialysate) such that the nanoparticles which become bioavailable during the first time period are sized and other otherwise configured such that they rapidly dissolve in interstitial tissue fluids so that they are quickly absorbed or otherwise taken up into the blood stream and the particle which become bioavailable in the second period dissolve more slowly in the interstitial fluids so they are not substantially absorbed into the blood stream until the beginning of the second period. In these and related embodiments, the first period may correspond to a period as soon as twenty minutes or as soon as ten minutes, or as soon as five minutes or even as soon as two minutes. The second period may correspond to a period as soon as 90 minutes, or as soon as 60 minutes, or as soon as 30 minutes, or as soon as 20 minutes, with longer and shorter periods contemplated. The biomodal particle size distributions (e.g. in terms of diameter or other particle dimension) to achieve these times and those for oral, parenteral and other forms of administration described herein can be determined using dose response and related pharmacokinetic methods known in the art. Particle size and particle distribution can be determined by a variety of methods and instruments known in the art including for example, optical microscopy and Dynamic Light Scattering (also known as Correlation Spectroscopy (PCS) or Quasi-Elastic Light Scattering (QELS) with suitable nanoparticle measurement instruments including those manufactured by Nanosight Ltd (NanoSight Ltd. Minton Park, London Road Amesbury, Wiltshire, SP4 7RT, United Kingdom) and TSI, Inc. (Shoreview, Minn.). Other instruments and methods are also contemplated.

In the production of nanonized iron particles by the RESS, SAS or PGSS methods, a number of parameters can be adjusted to regulate the size and/or size distribution of the iron-containing particles produced. For example, the solvent used in the supercritical fluid can be any one which provides the desired particle size, including any of a number of liquefied compressed gases known to the art such as gaseous oxides such as carbon dioxide and nitrous oxide; alkanes such as methane, ethane, propane, butane, and pentane; alkenes such as ethylene and propylene; alcohols such as methanol, ethanol and isopropanol; ketones such as acetone; ethers such as dimethyl or diethyl ether; esters such as ethyl acetate; halogenated compounds including sulfur hexafluoride, chlorofluorocarbons such as trichlorofluoromethane (Freon 11, CCl₃F,), dichlorofluoromethane (Freon 21, CHCl₂F), difluorochloromethane (Freon 22, CHClF₂), and fluorocarbons such as trifluoromethane (Freon 23, CHF₃); and elemental liquefied gases such as xenon. Optionally, the solvent can include mixtures of one or more suitable materials.

Additional parameters which can be varied during the RESS, SAS or PGSS methods to provide the desired size and/or size distribution of the iron-containing nanoparticles include, for example, concentration of the iron-containing compound in the supercritical fluids, temperature of the solution, pressure, and the diameter of the nozzle through which the supercritical solutions are forced (as in the RESS and PGSS methods).

Further, in one more embodiments of methods for making nanonized iron-containing compositions, including the RESS, SAS, and PGSS, the process can be adapted to make hollow and/or porous iron-containing nanoparticles, in one or more shapes including, for example, substantially spherical and tubular. Such hollow and/or porous nanoparticles can be configured to provide increased surface areas for contact with liquids in which the particles are dissolved (e.g. bodily fluids and/or aqueous solvents used for parenteral solutions) so as to increase rates and completeness of dissolution (which in turn, reduces bioavaibility times and reduces immune or other adverse reaction). Process parameters which can be modified to make such shapes and/or hollow/porous particles using RESS, SAS, PGSS or other methods can include for example, one or more of the nozzle pressure (through which the supercritical fluids are force), nozzle diameter, nozzle pressure and concentration of iron compound in the fluid. Further, one or more of these processes can be combined with solvent evaporation methods to produce hollow nanoparticles by evaporation of solvent within inside of the nanoparticles. In other embodiments, methods used to make fullerine-like hollow structures can be applied to make hollow iron-containing nano-sized particles having a spherical or other related shape. Such fullerine-like structures are described in “Fullerenes and related structures”, Ed. A. Hirsch, Springer Verlag 1999, pp. 189-234, or N. Grobert, Nachr. Chem. Tech. Lab., 47 (1999), 768-776, which is incorporated herein for all purposes.

In still other embodiments, one or more iron-containing compounds described herein, (e.g. ferric pyrophosphate, ferric chloride, ferrous citrate, etc.) can be fabricated into hollow nano sized tubes using hollow fiber fabrication methods such as extrusion spinning and electro spinning processes. Further description of the use of extrusion spinning and electro spinning processes and other methods for fabrication of hollow nano-sized tubes including porous nano tubes can be found U.S. Pat. No. 6,667,099 and U.S. Patent Publication No. 20060119015 (application Ser. No: 10/507,311), and U.S. Patent Publication No. 20110028834 (application Ser. No: 12/918,377) which are incorporated by reference herein for all purposes.

The length and other dimensions of various embodiments of iron-containing containing nanotubes can be configured to have various properties and/or perform one or more functions. For example in one or more embodiments, the iron-containing nanotubes can be configured to spin or otherwise be propelled in another manner by a magnetic force from an external magnet that is spinning or moving in another manner. This allows the nanotubes to act as magnetic spin bars when they are added to a solution containing nanonized iron particles to facilitate aseptic mixing of the solution so as to increase rates and completeness of dissolution of the nanonized iron including that of iron-containing nanotubes. The diameter to length ratio for achieving such spinning action or other desired movement of the nanotubes can be in the range of about 1:1.5 to about 1:20, with specific embodiments of 1:2, 1:3, 1:4, 1:5, 1:7, 1:8, 1:9, 1:10, 1:15, and 1:17 with the diameter of the nanotubes being 1000 nm or less, or 500 nm or less, or 250 nm or less, or 100 nm or less, or 50 nm or less, or 20 nm or less, or 10 nm or less. Also, depending upon the strength of the external magnet field used to produce spinning, the mass ratio of elemental iron to the total mass of the iron containing nano-particle (herein called iron to total mass ratio) can be selected to facilitate such spinning or other motion. Larger iron to total mass ratios can be used for magnetics having stronger magnetic fields and vice versa. In various embodiments, the iron to total mass ratio can be in the range of about 1:1 to 1:10, with specific embodiments of 1:2, 1:3, 1:4, 1:5, 1:6, and 1.7. For the case of ferric pyrophosphate, the iron to total mass ratio is about 1:3.

Other particle shapes, sizes and configurations of iron-containing nanoparticles described herein can also be configured (e.g.,. similar diameter, iron to total mass ratio, etc.) such that those particles can also be spun or otherwise propelled in an aqueous or other solution by an external magnetic force so that the spinning action or other movement of the particles can both help mix the solution and increase the rate and completeness of particle dissolution. Such embodiments also allow the mixing process to be done aseptically since no external magnetic mixing bar or other external mixing device must be added to the solution (and for that matter, later removed). In one or more particular embodiments, such spinnable/propellable particles can be used for a transdermal patch in which iron containing nanoparticles are mixed with an aqueous solution within or on the patch. After fluid is added to particles in the patch, the patch can be placed over a spinning magnet device (and/or the spinning magnet device can be turned on to start spinning) to spin the particles in solution and mix the solution. In one or more parenteral solution embodiments, embodiments of the spinnable iron containing nanoparticles can be aseptically added to an I.V bag or other container holding the parenteral solution and the bag then placed in proximity to a spinning magnet device so as to mix the solution and facilitate dissolution of the particles in solution.

Suitable iron compounds for use in nanonized iron-containing compositions can include, for example, ferric pyrophosphate, and related iron salts. Other suitable iron compounds can include without limitation, ferrous or ferric complexes comprising edetate, ethylenediamineedetate, ethylenediaminesuccinate, ferric citrate, ferric pyrophosphate citrate chelate, ferric ammonium citrate, ferric choline citrate, ferric manganese citrate, ferric quinine citrate, ferric sodium citrate, ferric formate, ferric gluconate, ferric ammonium sulfate, ferric ammonium oxalate, ferric potassium oxalate, ferric sodium oxalate, ferric trisglycinate, ferric bislycinate, ferrous saccharate, ferriecit, ferrous gluconate, ferrum vitis, ferrous hydroxide saccharate, iron-arene sandwich, acetylacetone iron salt, iron-dextran complex, iron-dextrin, iron-sorbitol-citric acid, saccharated iron oxide, ferrous fumarate, iron porphyrin, iron phtalocyamine, iron cyclam, dithiocarboxy-iron, desferrioxamine-iron, bleomycin-iron, ferrozine-iron, iron perhaloporphyrin, alkylenediamine-N,N′-disuccinic acid iron(III), hydroxypyridone-iron(III), aminoglycoside-iron, transferrin-iron, iron thiocyanate, iron complex cyanides, porphyrinato iron(III), polyaminopolycarbonate iron, dithiocarbamate iron, adriamycin iron, anthracycline-iron, MGD-iron, ferrioxamine B, ferrous citrate, ferrous sulfate, ferric gluconate complex, ferrous succinate, polyglcopyranosyl iron, polyaminodisuccinic acid iron, biliverdin-iron, deferiprone iron, ferric oxyhydride-dextran, dinitrosyl dithiolato iron, iron lactoferrin, 1,3-PDTA ferric complex salts, diethylenetriaminepentaacetic acid iron salts, cyclohexanediaminetetraacetic acid iron salts, methyliminodiacetic acid iron salts, glycol ether diaminetetraacetic acid iron complex, ferric hydroxypyrone, ferric succinate, ferric chloride, ferric glycine sulfate, ferric aspartate, sodium ferrous gluconate, ferrous hydroxide polymaltose, other pharmaceutically acceptable chelated iron complexes, blends, mixtures and/or combinations thereof.

In various embodiments employing nanonized iron compositions, such as ferric pyrophosphate, the therapeutically effective amount of iron delivered can be in the range from about 1 to about 300 mg, with specific embodiments of about 1 to about 100 mg, about 1 to about 50 mg, about 1 to about 25 mg, about 1 to 15 mg and about 3 to about 10 mg. Other ranges and specific amounts are also contemplated, e.g., greater than 100 mg, e.g., 500 mg. For embodiments employing ferric pyrophosphate, the amount of ferric pyrophosphate required to achieve these delivered doses is roughly three times the desired delivered dose, owing to the fact that iron makes up roughly one third of the molecular weight of ferric pyrophosphate. Though in various embodiments, this ratio can be adjusted depending upon the particular lot of ferric pyrophosphate or other factors. Such factors can include for example, the desired shelf life and the ambient conditions in which the ferric pyrophosphate is packed and/or stored including the humidity and temperature conditions. Higher ratios (e.g., 5:1, 7:1, 10:1, etc.) can be used to obtain longer shelf lives. Likewise, higher ratios can be used for higher temperature/and or humidity ambient storage conditions so as to maintain a desired delivered dose of iron over product shelf life.

Suitable routes of administration of the nanonized iron compositions may include, for example, oral, rectal, transmucosal, transdermal (either passive or active by such methods as iontophoresis), inhalation, nasal, or intestinal administration and parenteral delivery, including intramuscular, subcutaneous, intramedullary injections, as well as intrathecal, direct intraventricular, intravenous, intraperitoneal, intranasal, or intraocular injections. Parenteral administration includes delivery by intravenous injection, intramuscular injection, or dialysis, such as administration in a dialysate to a patient with chronic kidney disease. Such dialysate containing nanonized iron compositions can be used, for example, in peritoneal dialysis or hemodialysis. In some embodiments, nanonized iron compositions may be administered in a local rather than a systemic manner. For example, a suitable iron composition can be delivered via injection or in a targeted drug delivery system, such as a depot/reservoir or sustained release formulation used for transdermal or other administration. In related embodiments, the iron compositions may be configured to be delivered in both a local and systemic manner. In such embodiments, a nanonized iron composition having a first particle size (e.g., diameter) and size distribution can be used for systemic administration, and a second nanonized iron composition having a second particle size and size distribution can be used for local administration. In preferred embodiments, the first composition could have a smaller particle size than the second composition to allow for a fast bioavailable release of the first composition, and a slower longer term bioavailable release of the second composition.

The amount or dose of the nanonized iron composition to be administered can be determined based on the condition to be treated (e.g., iron deficiency vs. iron depletion, anemia, kidney disease, etc.) and patient weight, age, etc. Also, dosages can be based on known therapeutically effective doses for a particular condition that are delivered orally, intravenously, transdermally or by other delivery means (e.g., intranasally, inhalation, etc.) with adjustment for different absorption/uptake of the known method. For example, in the case of orally delivered iron-containing compounds (e.g., ferrous sulfate) for the treatment of anemia, typically 50 mg of elemental iron are delivered, of which only 10 to 25 mg are actually absorbed into the blood stream. Accordingly, the dose of elemental iron in the nanonized iron composition can be in the range of about 10 to about 25 mg or about 10 to about 50 mg with specific embodiments of 10, 15, 20, 25, 30, 40 and 50 mg. Additionally, in particular embodiments, dose response curves can be developed for the delivery of nanonized iron compositions using known pharmacological methods so as to determine specific doses of nanonized iron composition needed to deliver a desired dose of elemental iron. These methods can include measurement of various biomarkers of iron status in the body including one or both of serum iron concentrations as well as % transferrin iron saturation (herein transferrin iron saturation). Again, adjustment can be made for the weight and age of the patient as well as their particular condition, e.g., menstrual versus gestational anemia, iron deficiency versus iron depletion. Further, ongoing measurement of various iron status biomarkers can be used to titrate delivery of the nanonized iron composition over the course of a treatment or treatments. Such titration can include varying not only the dosage or amount of the nanonized iron composition, but also the particle size and particle size distributions. For example, over the course of a treatment using an intravenous solution or dialysate, the particle size of the nanonized iron composition can be increased over time so as to have a more delayed, longer term release of bioavailable iron.

In various embodiments, the particle size and particle size distribution of the nanonized iron compositions can be optimized for the particular route of administration in terms of various pharmacokinetic and related parameters such as bioavailability, rates of dissolution etc. For example, for intravenous delivery and/or dialysate solutions, the particle size and particle size distribution of the nanonized iron compositions may be chosen to achieve particular rates of dissolution and/or solubility. Smaller particle sizes and/or size distributions may be chosen to achieve faster rates of dissolution and higher solubilities. In particular intravenous and dialysate embodiments, the particle sizes (as determined by diameter or other particle dimension) of the iron-containing nanoparticles comprising the nanonized iron composition can be chosen to be in the 100 to 5000 nm range, the 500 to 10,000 nm range, the 1000 to 25,000 nm range, the 5000 to 50,000 nm range or the 10,000 to 100,000 nm range. Also, the particle size and particle size distribution can be optimized for other factors separately or in combination with rate of dissolution and/or solubility, such as stability, bioavailability and the presence of other compounds such as erythropoietin (or other red blood cell stimulating glycoprotein) or various preservatives such as citric acid.

In many embodiments, the particle size and/or particle size distribution of the nanonized iron compositions can be optimized to minimize the immune response of a patient to the iron compositions when those compositions are administered to patient in one or more forms described. This can include for example, when the iron-containing composition is administered orally, transdermally, intravenously and/or in a dialysate so as to increase and/or replenish an iron store of the patient. Such iron stores can include, for example, those found in the patient's red blood cells (e.g., in the form of hemoglobin) and that complexed to ferritin (e.g., that found in all cells of the patient's including the liver, spleen and bone).

The immune response of a given patient can be determined by a variety of means. For example, in one or more embodiments, immune response can be determined by monitoring the patient for development of a fever (also described herein as a pyrogenic reaction). The fever may be characterized as a low grade (e.g., body temperature (BT) in the range of about 100 to 101° F.), mid grade (BT in the range of about 101 to 103° F.), or high grade fever (BT in the range of about 103 to 104° F.) or other classification known in the medical arts. The grade of fever can be correlated (e.g., linear, first order, second order, logarithmic, etc.) to the dose of the nanonized iron compositions (using various mathematical methods such as least squares analysis, curve fitting etc.), with a score assigned to the immune response based on the grade of fever and the dose. Such scores can be used in the immune response profile described below. Since the scoring accounts for dosage, the patient need not be given a full clinical dose of the nanonized iron composition for purposes of assessing pyrogenic reaction or other immune response or adverse reaction. Also, a substitute composition can be used which is correlative/predictive of adverse reactions produced by iron compounds in the nanonized iron compositions but produces less severe affects. The scoring of pyrogenic reaction or other immune response can be adjusted to account for use of the substitute compound using correlations (e.g., linear, first order, second order, etc.) between the substitute and the iron compound the nanonized iron composition.

Further, in one or embodiments, the development of a fever may be used as an initial screen of immune response, with follow up for patients developing a fever using one or more methods described below. Such initial screening may also constitute a review of the patient's medical history and/or questioning of the patient for an immune response including pyrogenic reaction and others described herein to iron-containing compounds and/or compounds correlated with an immune response to the iron-containing compositions. Again, such correlations may be known or developed using methods described herein and/or known in the art.

As an adjunct or alternative to monitoring for pyrogenic reaction, the likelihood of the patient having an immune to embodiments of nanonized iron composition can also be determined by serological testing of the patient for the presence of antibodies to iron-containing compounds described herein or compounds known to be correlated to an immune response of such iron-containing compounds. Similar to pyrogenic reaction, such serological testing can also be used as an initial screen for immune response to embodiments of nanonized iron compounds with follow up with one or more tests described below. Also, similar to pyrogenic reaction, the serological testing results can be incorporated into the patient's immune response profile described below.

In yet other embodiments, immune response of a patient to embodiments of the nanonized iron compositions can be determined by the concentration and/or changes (e.g., an increase) in the concentration of the patient's T cells and B cells and associated levels of cytokines resulting from the administration of nanonized iron compositions or compounds determined to be correlated with immune response to such compositions. Such concentrations and/or concentration changes constitute biomarkers of immune response (BIR). Changes in various cellular concentrations such as T and B cells, macrophages, etc. constitute a subset of BIR's described herein as cellular biomarkers of immune response (CBIR). One more of these or other BIRs (e.g., antibody, C-reactive protein, etc.) can be quantitatively or qualitatively measured (using methods known in the art) before and after administration of nanonized iron compositions (or correlated compounds described above) having a particular particle size or range of particle sizes. A change in these or other related biomarkers of immune response can be used to quantify the level of the patient's immune response to a particular particle size or range of particles size of nanonized iron particles. Multiple BIR's can be pooled together by themselves and/or with other indicators of immune response (e.g. an increase in body temperature) to form an immune response profile for a given particle size or range of particle sizes of nanonized iron compositions. Such immune response profiles can be analyzed and used to screen patients who have or are likely to have an immune response to nanonized iron compositions so that a decision can be made to administer them nanonized iron-containing particles using one or more routes of administration described herein and/or known in the art. Further, for those patients who do have an immune response, the immune response profile can be used to select a particle size or a range of particles sizes of nanonized iron compositions which produce a minimal immune response for that patient. In other words, the particle size or range of particle sizes of iron compositions can be adjusted for a particular patient to minimize immune response for that patient.

In various embodiments for minimizing one or more immune responses (e.g., a pyrogenic reaction) or other adverse reactions (e.g. allergic reactions such as hives, swelling, a drop in blood pressure, slowed heart rate etc.) to various nanonized iron compositions described herein, the diameter (or other particle size dimension) of the iron-containing nanoparticles comprising the nanonized iron composition can be chosen to be less than 1000 nm, less than 500 nm, less than 250 nm, less than 50 nm, less than 25 nm or less than 10 nm Also, in these and related embodiments, the diameter (or other particle size dimension) of the iron-containing nanoparticles can be in the 10 to 20 nm range, the 10 to 100 nm range, the 50 to 250 nm range, the 100 to 500 nm range, the 250 to 1000 nm range, the 500 to 5,000 nm range, the 500 to 10,000 nm range, the 1000 to 25,000 nm range, the 5000 to 50,000 nm range or the 10,000 to 100,000 nm range. Also, in these and related embodiments exemplary nanonized compositions for minimizing immune response and other adverse reactions can include those in which 50% or more of the particles have a diameter (or other particle size dimension) within 1 to 1000 nm, or within 1 to 500 nm, or within 1 to 250 nm, or within 1 to 100 nm, or within 1 to 50 nm, or within 10 to 1000 nm, or within 10 to 500 nm, or within 10 to 250 nm, or within 10 to 100 nm, or within 10 to 50 nm, or within 50 to 1000 nm, or within 50 to 500 nm, or within 50 to 250 nm, or within 100 to 1000 nm, or within 100 to 500 nm, or within 100 to 250 nm. Adjustment in the above particle sizes and particle size ranges can be made for the particular route of administration, e.g., intravenous, oral, inhaled, transdermal etc.

Similar to the above embodiments, the particle size (as determined by diameter or other particle size dimension) and size distribution can also be optimized to minimize immune response (or other adverse reaction) in cases where the nanonized iron-containing particles are dissolved in an intravenous, or dialysate or other solution which includes the presence of other compounds such as various red blood cell stimulating glycoproteins, preservatives such as citric acid and any number of other therapeutic agents or other additives. Similar particle size and particle size distributions as mentioned above may be used in these and related embodiments with adjustment for the particular added compounds. For example, smaller particle sizes and/or particle size ranges can be used for added compounds which are more prone to illicit an immune response.

In still other embodiments, the particle size and particle size distribution of the nanonized iron compositions can be optimized to enhance red blood cell production for patients (e.g., renal disease or other dialysis patients), who are receiving red blood cell stimulating glycoproteins compounds in addition to the nanonized iron compositions. Such glycoproteins compounds can be delivered intravenously and/or in a dialysate used for dialysis. Exemplary red blood cell stimulating glycoproteins which can be used in these and related embodiments include erythropoietins and their analogues and related compounds. An example of an erythropoietin analogue includes peginesatide available from the Affymax Corporation, Palo Alto, California. Optimization of red blood cell production can be determined by measuring the patient's hematocrit as well as other hematological measurements and biomarkers known in the art. In these and related embodiments, the diameter (or other particle size dimension, e.g., length) of the iron-containing nanoparticles comprising the nanonzied iron composition can be chosen to be less than 1000 nm, less than 500 nm, less than 250 nm, less than 50 nm, or less than 10 nm. Also, in these and related embodiments, the diameter (or other particle size dimension, e.g., length) of the iron-containing nanoparticles can be in the 10 to 100 nm range, the 50 to 250 nm range, the 100 to 500 nm range, the 250 to 1000 nm range, the 500 to 5,000 nm range, the 500 to 10,000 nm range, the 1000 to 25,000 nm range, the 5000 to 50,000 nm range or the 10,000 to 100,000 nm range. Also, in these and related embodiments exemplary nanonized compositions include those in which 50% or more of the particles have a diameter (or other particle size dimension) within 1 to 1000 nm, or within 1 to 500 nm, or within 1 to 250 nm, or within 1 to 100 nm, or within 1 to 50 nm, or within 10 to 1000 nm, or within 10 to 500 nm, or within 10 to 250 nm, or within 10 to 100 nm, or within 10 to 50 nm, or within 50 to 1000 nm, or within 50 to 500 nm, or within 50 to 250 nm, or within 100 to 1000 nm, or within 100 to 500 nm, or within 100 to 250 nm. Further, the particle size (e.g., as measured by diameter or other particle size dimension) and size distribution can also be optimized to enhance red blood cell production where the intravenous injection or dialysate (or other delivery medium, e.g., transdermal solutions, inhalation solution, etc.) includes other compounds such as various preservatives (e.g., citric acid) and any number of other therapeutic agents or other additives. Similar particle size and particle size distributions as mentioned above may be used in these and related embodiments.

In various embodiments for minimizing adverse GI reactions associated with oral delivery of iron-containing compounds (e.g., cramping, diarrhea, constipation) and/or improving iron absorption in the small intestine, the diameter (or other particle size dimension) of the iron-containing nanoparticles comprising the nanonized iron composition can be chosen to be less than 1000 nm, less than 500 nm, less than 250 nm, less than 50 nm, less than 25 nm or less than 10 nm Also, in these and related embodiments, the diameter (or other particle size dimension) of the iron-containing nanoparticles can be in the 10 to 20 nm range, the 10 to 100 nm range, the 50 to 250 nm range, the 100 to 500 nm range, the 250 to 1000 nm range, the 500 to 5,000 nm range, the 500 to 10,000 nm range, Also, in these and related embodiments exemplary nanonized compositions for minimizing adverse GI reactions and improving absorption in the small intestine can include those in which 50% or more of the particles have a diameter (or other particle size dimension) within 1 to 1000 nm, or within 1 to 500 nm, or within 1 to 250 nm, or within 1 to 100 nm, or within 1 to 50 nm, or within 10 to 1000 nm, or within 10 to 500 nm, or within 10 to 250 nm, or within 10 to 100 nm, or within 10 to 50 nm, or within 50 to 1000 nm, or within 50 to 500 nm, or within 50 to 250 nm, or within 100 to 1000 nm, or within 100 to 500 nm, or within 100 to 250 nm.

In various embodiments, the size of the iron-containing nanoparticles comprising the nanonized iron compositions can be selected such that when the iron-containing nanoparticles are added to a parenteral or other solution for administration to the patient, the particles dissolve sufficiently in the solution so that no filtering of the solution is needed to prevent an immune response such as a pyrogenic reaction due to any un-dissolved particles which get into the patient's blood stream or other location triggering an immune response. In various embodiments for achieving this, the diameter (or other particle size dimension) of the iron-containing nanoparticles can be 1000 nm or less, or 500 nm or less, or 250 nm or less, or 100 nm or less, or 50 nm or less, or 20 nm or less, or 10 nm or less. Related embodiments for achieving this result provide nanonized iron compositions in which 50% or more of the particles have a diameter within 1 to 1000 nm, or within 1 to 500 nm, or within 1 to 250 nm, or within 1 to 100 nm, or within 1 to 50 nm, or within 10 to 1000 nm, or within 10 to 500 nm, or within 10 to 250 nm, or within 10 to 100 nm, or within 10 to 50 nm, or within 50 to 1000 nm, or within 50 to 500 nm, or within 50 to 250 nm, or within 100 to 1000 nm, or within 100 to 500 nm, or within 100 to 250 nm.

As is discussed herein, various embodiments of the invention provide nanonized iron compositions which can dissolve in a parenteral or other solution delivered to a patient receiving treatment for iron deficiency. In particular embodiments, the nanonized iron compositions can be configured to dissolve in an aqueous solution disposed in a transdermal patch used for passive or active transport (e.g., by iontophoresis) of the iron composition across the skin of a patient being treated for iron deficiency. Further, the nanonized iron compositions can be configured to dissolve in situ in the patch to achieve a selected iron concentration within the patch (for delivery of a desired dose of elemental iron) when the aqueous solution is added to or released within the patch and to do so with minimal or no mixing of the solution. In various embodiments for achieving this, the diameter (or other particle size dimension) of the iron-containing nanoparticles can be 1000 nm or less, or 500 nm or less, or 250 nm or less, or 100 nm or less, or 50 nm or less, or 20 nm or less, or 10 nm or less. Related embodiments for achieving this result provide nanonized iron compositions in which 50% or more of the particles have a diameter within 1 to 1000 nm, or within 1 to 500 nm, or within 1 to 250 nm, or within 1 to 100 nm, or within 1 to 50 nm, or within 10 to 1000 nm, or within 10 to 500 nm, or within 10 to 250 nm, or within 10 to 100 nm, or within 10 to 50 nm, or within 50 to 1000 nm, or within 50 to 500 nm, or within 50 to 250 nm, or within 100 to 1000 nm, or within 100 to 500 nm, or within 100 to 250 nm.

Various embodiments of the invention can provide instructions for administering one or embodiments of nanonized iron compositions to a patient via one or more routes of administration (e.g., intravenous, intramuscular, dialysate, oral, transdermal patch, inhaled etc.). Embodiments of the invention can also provide instructions for screening patients for any immune response to embodiments of the nanonized iron compositions described herein using methods described herein (e.g., testing for BIRs and CBIR's, serological tests, body temperature measurement and/or use of an immune response profile) as well as others known in the art. Further, embodiments of the invention can also provide instructions for selecting nanonized iron compositions which minimize any immune response or other adverse reaction of patients both screened and unscreened. Embodiments of such instructions can also include instructions for selection and/or adjustment of the particle size (e.g., as measured by diameter or other dimension) or particle size range of the nanonized iron compositions to minimize immune response. One or more embodiments of such instructions (e.g., for screening for immune response and/or selecting nanonized iron compositions to minimize it) can be packed together with one or embodiments of nanonized iron compositions described herein so as to comprise a kit for the administration of nanonized iron compositions to a patient in need thereof. In some embodiments, the kit can comprise a plurality of groups of nanonized iron compositions, with each group including nanonized iron compositions having a different particles size or particle size range. Typically, the groups of nanonized iron compositions will be kept in separate containers (e.g., bottles, vials, ampules, syringes, etc.), but not necessarily. In use, such kit embodiments provide a method for the medical professional to select a particle size or particle size range of nanonized iron compositions which minimizes immune response to such compositions. Further, the instructions in such kit embodiments may also provide directions to the pharmacist or other medical professional as to how to select and mix the nanonized iron compositions from the containers so as to produce a resulting composition having a particle size range which minimizes immune response. In use, such kit embodiments provide a method for selecting and preparing nanonized iron compositions which minimize immune response in a patient to such compositions. For intravenous, dialysate and other forms of parenteral administration, after selection of the desired nanonized iron composition (or compositions), the instructions can also provide directions to a pharmacist or other medical professional as to how to prepare a solution of the selected composition(s) for administration to the patient (e.g., direction on concentration and dosage).

### CONCLUSION

The foregoing description of various embodiments of the invention has been presented for purposes of illustration and description. It is not intended to limit the invention to the precise forms disclosed. Many modifications, variations and refinements will be apparent to practitioners skilled in the art.

Elements, characteristics, or acts from one embodiment can be readily recombined or substituted with one or more elements, characteristics or acts from other embodiments to form numerous additional embodiments within the scope of the invention. For example, nanonized iron compositions having one particle size or particle size distribution can be readily combined with those having a different particle size or particle size distribution. Similarly, nanonized iron compositions comprising one iron-containing compound, such as ferric pyrophosphate, can be combined with nanonized iron compositions comprising one or more other iron-containing compound such as ferric chloride, ferrous citrate or ferrous sulfate. Moreover, elements that are shown or described as being combined with other elements, can, in various embodiments, exist as stand-alone elements. Hence, the scope of the present invention is not limited to the specifics of the described embodiments, but is instead limited solely by the appended claims.

1. A composition comprising nanoparticles of an iron-containing compound for the treatment of iron deficiency in a patient, the nanoparticles having a particle size or particle size range which minimizes an adverse reaction of a patient to administration of the iron-containing compound to increase an iron store in the patient.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A kit for administering nanoparticles of an iron-containing compound to a patient for the treatment of iron deficiency, the kit comprising:
a composition of claim 1; and
instructions for administering the composition for the treatment of iron deficiency.

54. A kit for administering nanoparticles of an iron-containing compound to a patient for the treatment of iron deficiency, the kit comprising:
a plurality of groups of nanoparticles of the iron-containing compound, each group including nanoparticles having a different particle size or different range of particle sizes from that in other groups; and
instructions for selecting from the plurality of groups of nanoparticles of the iron-containing compound, nanoparticles having a particle size or range of particle sizes which minimize an immune response of the patient to the iron-containing compound.

55. (canceled)

56. (canceled)

57. A method of treating iron deficiency comprising administering to a patient in need thereof a therapeutically effective amount of a composition of claim 1.

58. (canceled)

59. (canceled)

60. (canceled)

61. A method of treating a patient who has iron deficiency, the method comprising:
selecting a patient who has iron deficiency;
administering to the patient a composition comprising nanoparticles of an iron-containing compound, wherein the particle size of the nanoparticles of the iron-containing compound is selected to minimize an adverse reaction of the patient to the iron-containing compound; and
increasing an iron store of the patient with minimal adverse reaction of the patient to the iron-containing compound.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. A method of treating anemia in a patient who has kidney disease, the method comprising:
selecting a patient who has kidney disease and anemia; and
administering to the patient a composition comprising nanoparticles of an iron-containing compound and a glycoprotein which stimulates red blood cell production, wherein the particle size of the iron-containing compound is selected to enhance red blood cell production stimulated by the glycoprotein.

93. The method of claim 92, wherein the iron-containing compound comprises ferric pyrophosphate.

94. The method of claim 92, wherein the glycoprotein comprises erythropoietin.

95. The method of claim 92, wherein the glycoprotein comprises peginesatide.

96. The method of claim 92, wherein the iron-containing nanoparticles have a diameter of about 1,000 nm or less.

97. The method of claim 92, wherein the iron-containing nanoparticles have a diameter of about 500 nm or less.

98. The method of claim 92, wherein the iron-containing nanoparticles have a diameter of about 250 nm or less.

99. The method of claim 92, wherein the iron-containing nanoparticles have a diameter of about 100 nm or less.

100. The method of claim 92, wherein the iron-containing nanoparticles have a diameter of about 50 nm or less.

101. A method of treating iron deficiency anemia in a patient, the method comprising:
selecting a patient who has iron deficiency; and
administering to the patient a composition comprising nanoparticles of an iron-containing compound wherein the nanoparticles have a first particle size for achieving a first level of bioavailability of the iron-containing compound in the patient in a first time period and a second particle size for achieving a second level of bioavailability of the iron-containing compound in the patient in a second time period, wherein the first time period is shorter than the second time period.

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. A method of treating a patient who has iron deficiency, the method comprising:
selecting a patient who has iron deficiency;
screening the patient for an adverse reaction to an iron-containing compound;
based on the screening, selecting a composition comprising nanoparticles of an iron-containing compound, wherein a particle size of the iron-containing compound is selected to minimize an immune response of the patient to the iron-containing compound; and
administering the composition comprising the nanoparticles of iron-containing compound to the patient; and
increasing an iron store of the patient with minimal adverse reaction of the patient to the iron-containing compound.

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. A method of treating a patient who has iron deficiency, the method comprising:
selecting a patient who has iron deficiency;
providing a composition comprising nanoparticles of an iron-containing compound for the treatment of iron deficiency in the patient, the nanoparticles having a particle size or particle size range which minimizes an adverse reaction of the patient to administration of the iron-containing compound to increase an iron store in the patient; and
providing instructions for administering the composition for the treatment of iron deficiency so as to increase an iron store of the patient while minimizing an adverse reaction of the patient to administration of the iron-containing compound.

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. A method for selecting treatment for a patient having iron deficiency, the method comprising:
selecting a patient who has iron deficiency;
providing a plurality of groups of nanoparticles of an iron-containing compound for treatment of iron deficiency, each group of nanoparticles including nanoparticles having a different particle size or different range of particle sizes from that in other groups; and
providing instructions for selecting from the plurality of groups of nanoparticles of the iron-containing compound, nanoparticles having a particle size or range of particle sizes which minimize an adverse reaction of the patient to the iron-containing compound.

149. (canceled)

150. (canceled)

151. (canceled)

152. (canceled)

153. (canceled)

154. (canceled)

155. (canceled)
